(19) United States
(12) Patent Application Publication
(10) Pub. No.: US 2012/0226145 A1
(43) Pub. Date: Sep. 6, 2012

(54) TRANSCUTANEOUS ROBOT-ASSISTED ABLATION-DEVICE INSERTION NAVIGATION SYSTEM

(75) Inventors: Stephen Kin Yong Chang, Singapore (SG); Chee Kong Chui, Singapore (SG)

(73) Assignee: National University of Singapore, Singapore (SG)

(21) Appl. No.: 13/407,307

(22) Filed: Feb. 28, 2012

Related U.S. Application Data
(60) Provisional application No. 61/448,829, filed on Mar. 3, 2011.

(51) Int. Cl. A61B 18/18; A61B 6/00

(52) U.S. Cl. 600/424

(57) ABSTRACT

A robotic system for overlapping radiofrequency ablation (RFA) in tumor treatment is disclosed. The robot assisted navigation system is formed of a robotic manipulator and a control system designed to execute preoperatively planned needle trajectories. Preoperative imaging and planning is followed by interoperative robot execution of the ablation treatment plan. The navigation system combines mechanical linkage sensory units with an optical registration system. There is no requirement for bulky hardware installation or computationally demanding software modules. Final position of the first needle placement is confirmed for validity with the plan and then is used as a reference for the subsequent needle insertions and ablations.

### RELATED APPLICATIONS

This application claims the benefit of U.S. Provisional Application No. 61/448,829 filed on Mar. 3, 2011. The entire teachings of the above application are incorporated herein by reference.

### FIELD OF THE INVENTION

Disclosed is a system proposed to enhance the effectiveness of computer aided surgery with a robot assisted needle insertion device in transcutaneous procedures.

### BACKGROUND OF THE INVENTION

Current practice for transcutaneous radiofrequency (RF) ablation involves a series of imaging and manual insertion based on the judgment of radiologists or surgeons. This method is tedious and often subject to uncertainties especially in the case of a large tumor where multiple needle insertions are required to destroy the entire tumor. Despite the development of sophisticated imaging modalities and surgical planning software, the bottleneck continues to be the uncertainty associated with intraoperative execution especially without real time image guidance.

While experienced surgeons may perform ablation therapy using real time ultrasound image guidance, this method has its disadvantages including the inadequacy in depth perception and the creation of a transient hyperechoic zone due to the microbubbles in ablated tissue.

### SUMMARY OF THE INVENTION

The present invention addresses the foregoing problems in the art. In particular, a system and method of the invention provide robotic assistance and navigational guidance for needle placement in an overlapping ablation technique during tumor treatment. Example tumor areas effectively treated by the present invention may be one tumor of >3 cm diameter (for example), or multiple tumors near each other (each about 2-3 cm diameter for example) totaling 4 cm or more diameter target area, or other tumorous areas (formed of one or many localized tumors) of an effective size or volume requiring multiple ablations. In the prior and state of the art, multiple ablations of such relatively large tumor areas are difficult to perform accurately throughout and have no effective monitoring method. Hence embodiments of this invention present a solution to overcome these difficulties.

The present invention procedure of preoperative imaging and planning followed by intraoperative robotic execution of the ablation treatment plan is more systematic and consistent compared to the existing judgment based approach. A specialized robotic mechanism and control system are designed to execute preoperatively planned needle trajectories. Its navigation system combines mechanical linkage sensory units with an optical registration system. There is no demanding requirement for bulky hardware installation or expensive computational software modules.

The invention method and system can readily operate in any conventional operating theater. Apart from task requirements of the system, the apparatus also contains functional features that address safety and compatibility requirements in a surgical environment. In addition, the elegant design facilitates potential expansions. The application of orientation-transport and micro-macro manipulator design concepts makes the mechanism adaptive to the execution of complicated preoperative plans required to perform the overlapping ablation technique.

Two ex-vivo experiments and an in-vivo study are conducted with the developed prototype TRAINS, herein referenced as the preferred embodiment.

Embodiments (methods and systems of computer-aided surgery having robot assisted needle insertion) comprise: preoperatively imaging a target tumor area and planning target needle positions, resulting in a preoperative treatment plan;
using a 3D coordinate tracking system, translating the preoperative treatment plan into intraoperative execution, i.e., a joint trajectory plan having robotic paths of operation; and
robotically executing transcutaneous insertion of a needle following the robotic paths of the joint trajectory plan.

The preoperative treatment plan has target needle positions in image coordinates. The step of translating thus (i) transforms target needle positions to real world coordinates and (ii) digitally computes a joint trajectory plan by converting a design specific kinematic model (for treating the target tumor area) to robotic paths.

The step of robotically executing is by a robotic manipulator and the 3D coordinate tracking system. This step includes confirming validity of placement of a first needle insertion (final position) and once confirmed, using the first needle placement (final position) as the reference for subsequent needle insertions and ablations of the joint trajectory plan.

### BRIEF DESCRIPTION OF THE DRAWINGS

The foregoing will be apparent from the following more particular description of example embodiments of the invention, as illustrated in the accompanying drawings in which like reference characters refer to the same parts throughout the different views. The drawings are not necessarily to scale, emphasis instead being placed upon illustrating embodiments of the present invention.

FIG. 1 is a schematic view of one embodiment of the present invention, namely the preferred embodiment hardware setup.

FIGS. 2a-2b are flow diagrams of data, control and operation of the embodiment of FIG. 1.

FIGS. 3 and 6 are respective schematic views of the robotic manipulator in the embodiment of FIG. 1.

FIG. 4 is a graphical illustration of the kinematics model of the manipulator system in embodiments.

FIG. 5 is a schematic view of a submanipulator design in the robotic manipulator of FIGS. 3 and 6.

### DETAILED DESCRIPTION OF THE INVENTION

A description of example embodiments of the invention follows. The teachings of all patents, published applications and references cited herein are incorporated by reference in their entirety.

The present invention introduces a transcutaneous robot assisted insertion navigation system that serves to bridge the gap in preoperative planning and intraoperative procedure by translating the preoperative plan into intraoperative execution consistently.

The preferred embodiment, the Transcutaneous Robot-assisted Ablation-device Insertion Navigation System (TRAINS) 100 is described now with reference to the Figures. Specific industrial applications include (but are not limited to): transcutaneous procedure for ablation therapy and needle placement in interventional procedures (including ablation therapy, biopsies, and laparoscopic procedures). Other applications are in the purview of those skilled in the art given this disclosure.

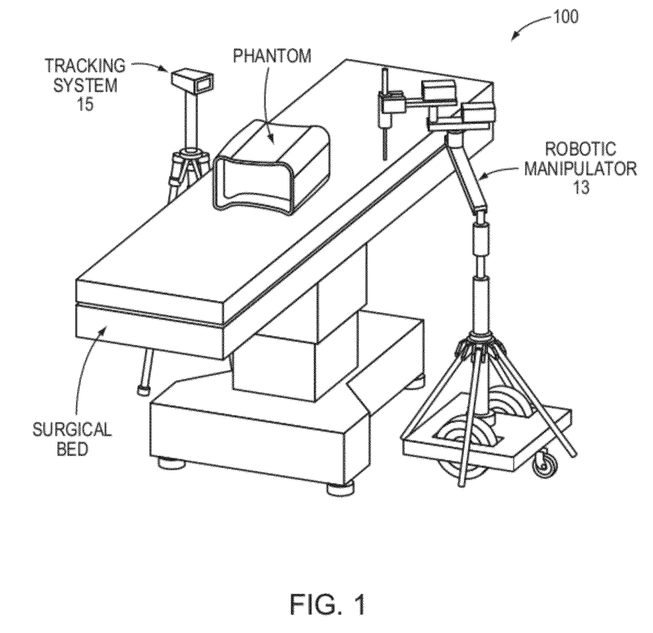

TRAINS is a system 100 for performing a transcutaneous procedure for ablation treatment in relatively large tumor areas (for non-limiting example, on the order of 2-3 cm diameter, formed of one or multiple close to each other tumors) through a preplanned robotic execution based on image diagnosis and a preoperative surgical plan. The invention system 100 comprises a robotic manipulator system 13 and a 3D coordinate tracking system 15 as illustrated in FIG. 1. To achieve consistent and precise results with multiple overlapping ablations, the invention system 100 applies the robotic manipulator system 13 to execute multiple needle insertions with pre-determined insertion trajectories. Validity of the first needle placement (final position) is confirmed before proceeding with subsequent needle insertions. The confirmed first needle placement (final position) is then used as the reference for the subsequent needle insertions and ablations in the multiple ablations intraoperative execution of the surgical plan.

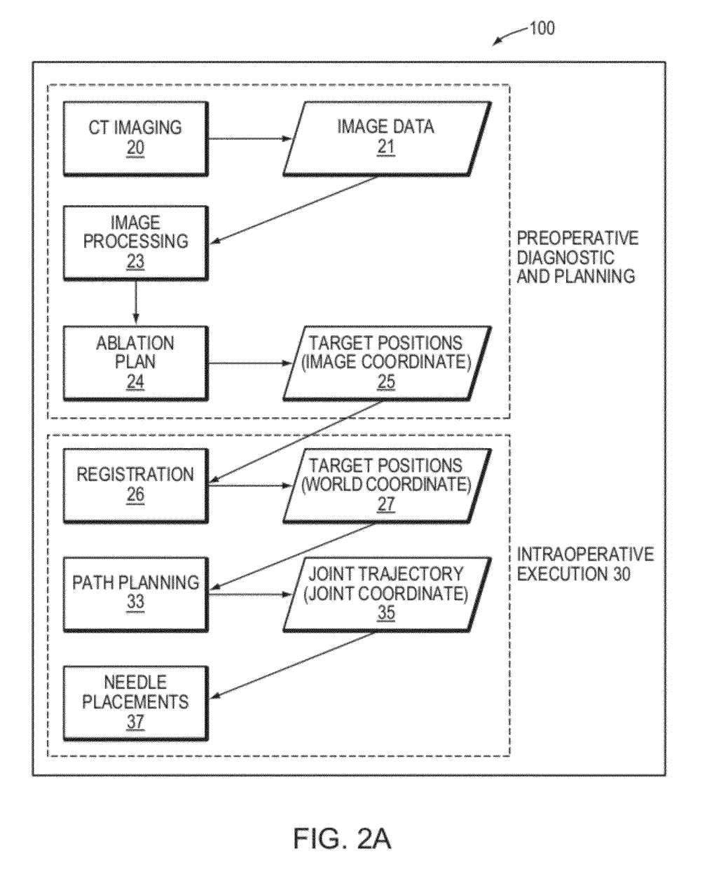

A systematic approach to be executed with the developed prototypical system 100 is illustrated in FIGS. 2a-2b. First, diagnostic images (e.g., CT imaging, MRI, or the like) 20 of a subject patient are taken. The resulting image data 21 are subsequently processed with image processing techniques 23 (common or known in the art) for tumor segmentation and identification. With the visual geometry of the subject tumor area obtained, a clinician can perform the appropriate planning 24 for the ablation treatment. For instance, a 3D reconstructed image is created using available visualization software. The three dimensional image(s), together with a needle insertion and ablation simulation user interface, enable the surgeon to plan the needle trajectories in order to achieve safe and complete ablation of the subject tumor area (which may be formed of one or many localized tumors, and which may amount to a sufficiently large size or volume that requires multiple ablations).

Next, the planned needle trajectories and ablation target positions computed in image coordinates 25 are mapped to real-world coordinates 27 through registration 26. Known registration techniques are used. The resulting target (real-world coordinates) data is used at computerized path planning step or module 33 to compute a joint trajectory 35 based on the design specific kinematic model (detailed later). Eventually the robotic manipulator 13 executes the preplanned path for needle placement 37 and delivers ablation therapy.

More specifically continuing from FIG. 2a to FIG. 2b, a computerized path plan 33 for needle placement 37 is registered 26 with the robotic system 13 in an intra-operative setting. This initiates computer supervised control execution 30 of multiple needle insertions (for non-limiting example for treatment of relatively large tumor areas). With the registration and computerized path plan 33 confirmed, an RF needle 47 is inserted into the target region (tumor) in the subject organ using the robotic system 13. The final position of first needle placement 37 in the organ is confirmed (validated) via existing/known imaging modality such as ultrasound 39. Ablation proceeds, and robotic system 13 proceeds with the second and subsequent needle insertions and ablations of the registered plan 33 using the confirmed and validated final position of the first needle placement 37 as a reference. To accomplish this, marker(s) may be deployed on the surface of the patient, and registered with the computer system to serve as reference for subsequent insertions and calibration if needed, as well as modification of surgical plan. Marker referencing techniques common in the art may be utilized.

An alternative embodiment is that after the final position of first needle placement 37 in the organ is confirmed (validated) via existing/known imaging modality such as ultrasound 39, a marker is placed at the target position prior to or after the ablation of the first needle placement 37. The marker may be an ultrasound contrast medium that can be injected into the tissue or other material that can be tracked external to the body. Robotic system 13 proceeds with the second and subsequent needle insertions and ablations of the registered plan 33. The marker may serve as the reference for subsequent insertions and calibration if needed as well as modification of surgical plan.

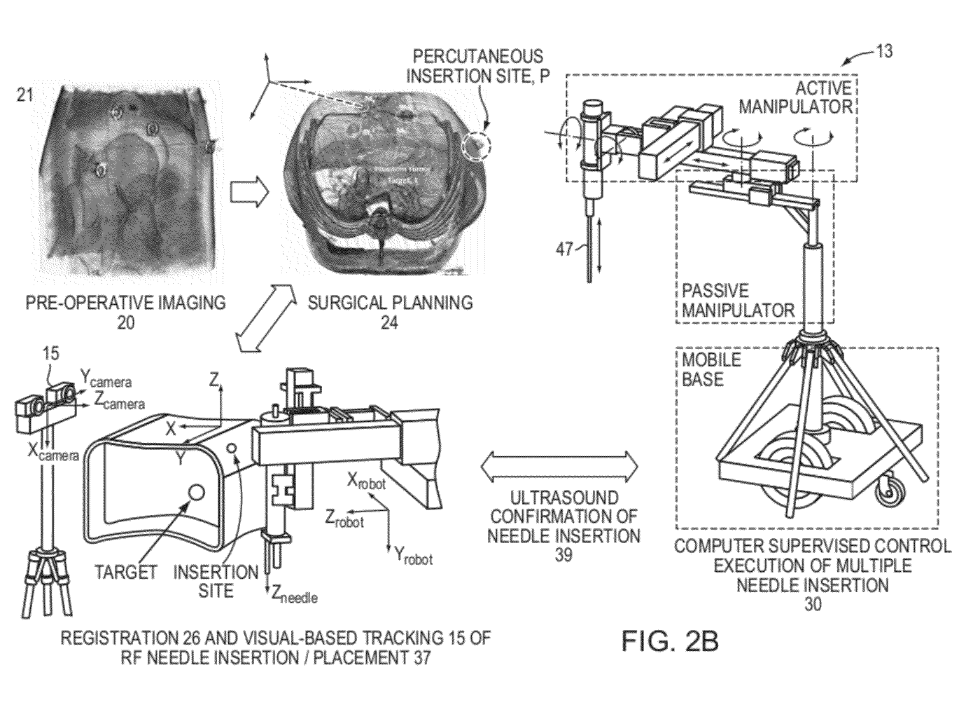

The robotic system 13 includes a novel manipulator mechanism together with its motion control software for execution of needle manipulation and insertion trajectories. FIG. 3 shows the developed prototype of the manipulator 13. There is a main manipulator unit or arm 41 and a submanipulator assembly 43. The subassembly 43 includes an end-effector 45 holding the RF needle 47. The preplanned kinematics trajectories of 33 (FIG. 2a) are based on preoperative medical images and a surgical plan conducted by the radiologists and surgeons. Developed software modules present, via a computer, a visual display of a 3D tumor model. Subsequently, the desired path input design by the surgeon is interpreted and converted (by the computer) to a robotic path in the joint domain of 35. These joint path trajections 35 are then executed intraoperatively by the robot assisted navigation system 100. For example, the resulting joint path trajections 35 are programmed into or otherwise operatively communicated to robotic manipulator 13 (having a digital processor therein) for intraoperative execution. Various digital processing (programmable processor, ASICS, FPGA's, etc.) and communication means (wide area computer networks, local area networks, Bluetooth, Wi-Fi or other wireless or wire/cable networks and protocols and so forth) in the art are employed.

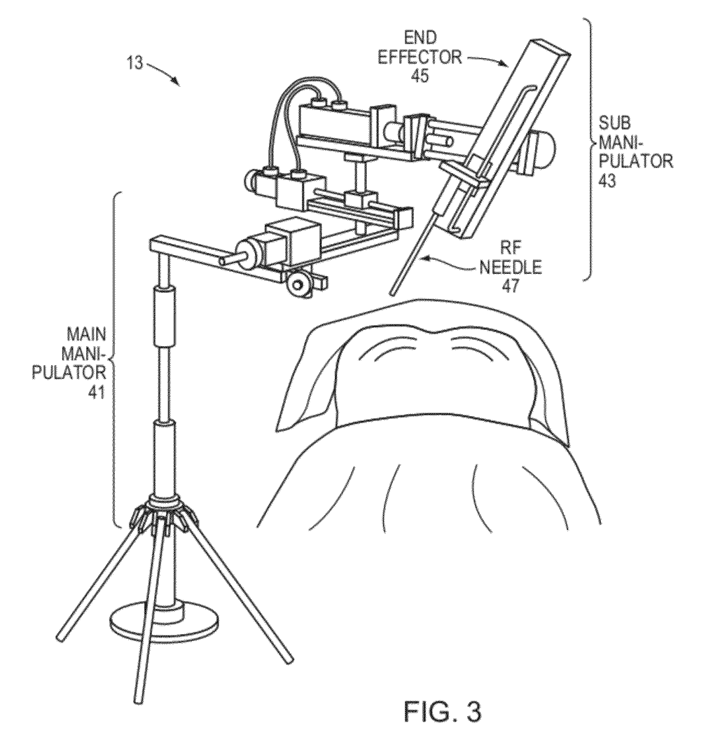

In a preferred embodiment, the specialized robotic arm (main manipulator unit) 41 is an 8 degree of freedom (DOF) serial manipulator comprising three passive links and five motorized axles. It applies the concept of a micro-macro manipulator for rapid deployment and fine placement of the needle. FIG. 4 shows the kinematic architecture of the manipulator 13. The first three passive links (subscripts 0, 1, 2, 3) make up the main manipulator unit 41 responsible for deployment of the needle 47 to an initial position. The main manipulator unit 41 is designed in a selective compliant assembly robot arm (SCARA)-like configuration which manipulates the submanipulator system 43 (at the distal end) in a cylindrical workspace. Link 4 and beyond constitute the submanipulator system 43. The 5-DOF (subscripts 4, 5, 6, 7, 8) submanipulator system 43 has an X-Y translator portion (shown at the beginning of l₄ in the upper portion of FIG. 4) coupled to a needle guiding unit (shown at subscripts 5, 6, 7, 8 in the lower portion of the figure). Submanipulator system 43 manipulates precise motion of the needle guiding unit (including end effector 45) within the submanipulator workspace and is capable of dexterous manipulation.

In addition to the geometric optimization, this architecture also facilitates a more precise positioning during reinsertion of needles for multiple overlapping ablations. The translational approach in regional structure facilitates the implementation of a high-precision translational stage for joints (links) 4 and 5.

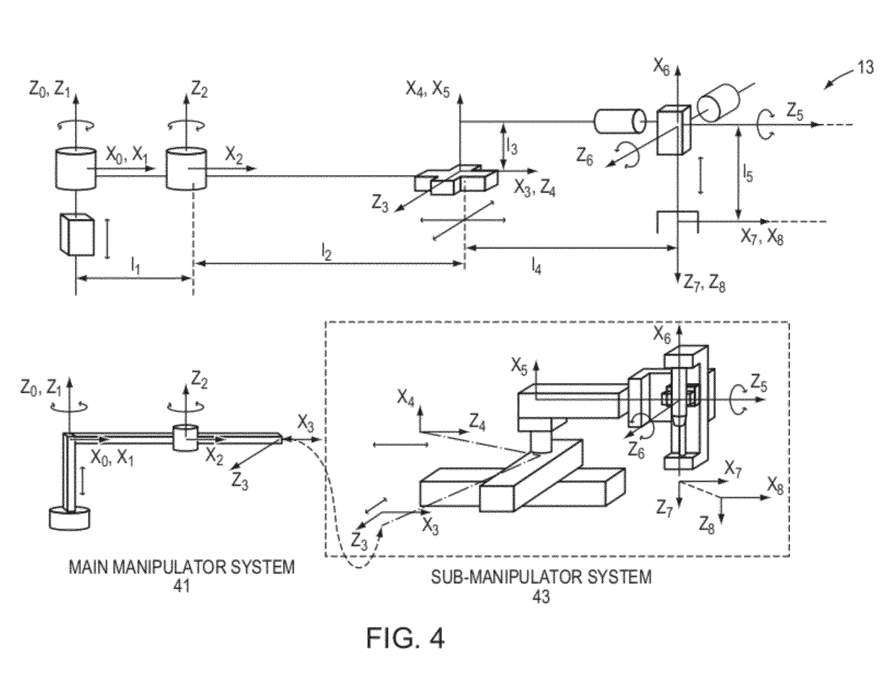

The complete design of the submanipulator assembly 43 in one embodiment is featured in FIG. 5. Shown are two outboard joints (the orientation structure) 51 and two inboard joints 55 (the transport structure). The two outboard joints 51 provide a wrist interface with two orthogonal revolute joints that orient the needle-driving end effector 45. The two inboard joints 55 provide a high-precision planar translational stage that manipulates the end effector 45 in a planar workspace. This design facilitates remote center of motion (RCM). RCM is a kinematic feature that pivots surgical tools on a constrained point during a minimally invasive procedure. In a more technical definition, the RCM mechanism decouples rotation and translation motion of the surgical tools remotely from the robotic end effector 45. This enables multiple needle 47 insertions through a single port during laparoscopic surgery. While the RCM can be maintained through software control, the submanipulator 43 design also accommodates mechanical compliance of RCM.

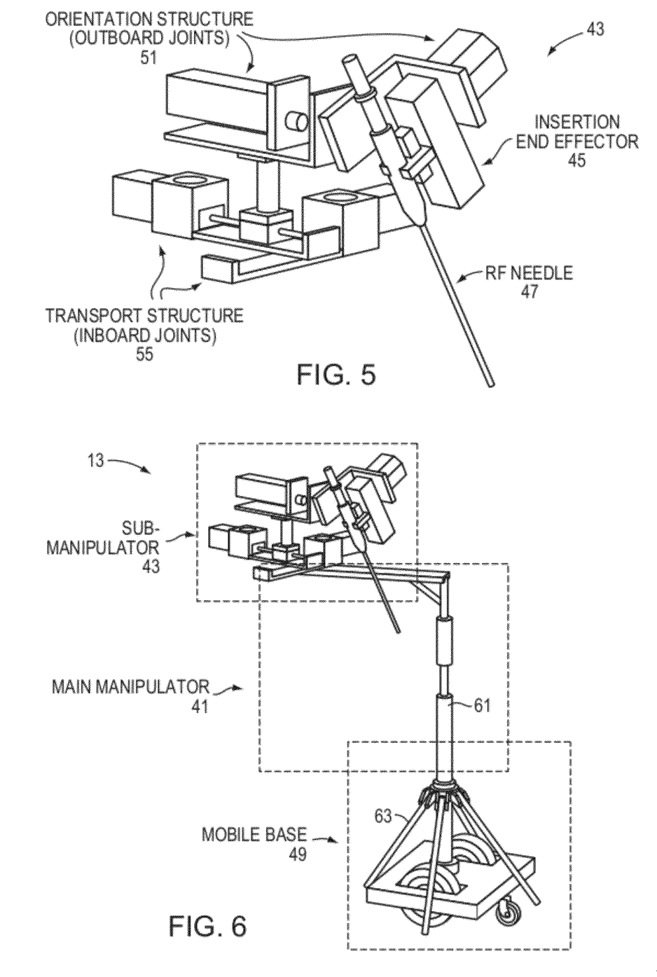

The entire robotic manipulator 13 can be mounted on to a wheeled mobile base 49 as showed in FIG. 6. Once the mobile base 49 is at the desired position, support stands or legs 63 at the base stem 61 can be deployed to ground the base firmly. The supporting point is designed to vary along the base stem 61 so that the area of the base 49 is independent of the elevation of the manipulator 13. The manipulator 13 can also be fixed onto a standard surgical bed along the rail.

The articulating nature of the manipulator 13 structure is advantageous for navigational trackers implementation. Wireless orientation sensors or encoders can be used for tracking the serial manipulator. Task coordinates can be obtained easily from joint coordinates 35 and vice versa by applying the appropriate Jacobian transformation. The closed form kinematic and dynamic model can be established. This is made possible by the design which partitions the multi-objective task of coarse needle transport, fine needle positioning and incision orientation. Hence, there is minimal need for demanding real time data acquisition and numerical computation of inverse kinematics. The invention system 100 uses an optical coordinate tracker 15 (FIG. 1) to establish spatial awareness of the surgical environment. Once registration 26 (via common techniques) of the relevant entities is done, the robotic manipulator 13 is able to navigate through its joint sensors (mechanical linkage sensory units). Applicant's adopted a generic rigid transformation approach for registration and implemented manipulator 13 with a neat wireless optical triangulation system during an animal study. This is a non invasive extrinsic approach based on a fiducial skin marker. Alternatively a vision based tracking unit can be used for the same purpose.

Embodiments of the present invention, such as preferred embodiment TRAINS 100, are resource efficient as each is specifically designed for transcutaneous ablation therapy. An embodiment 100 can be installed readily in any conventional operating theater or imaging center for outpatient surgery. Commercially available robotic systems capable of MIS do not serve as an efficient solution to the issue of RF ablation for relatively large tumor area treatment. As a result of the functional varieties of existing commercial surgical robotic systems, a larger operation envelope and more manpower are required to set up and operate them. It is therefore unpractical to utilize existing robotic surgical systems for transcutaneous procedures.

While there are many registration methods, tracking apparatus and surgical navigation systems, most of these operations are passive and involve manual control. They provide a form of spatial guidance but do not guarantee effective and consistent translation of preoperative plans to intraoperative executions. Applicant's introduction of robotic assistance in TRAINS 100 addresses this issue. Robotic execution of transcutaneous insertion ensures that multiple needle placement is consistent with the preplanned target.

The use of mechanical linkage sensory units and a vision based registration system 15 (discussed above) in tandem for surgical navigation is advantageous in terms of consistency, robustness and cost efficiency. This approach avoids the need to install expensive modalities like ultrasound localizers or high precision 3D vision systems which may not be available in a conventional operating theater.

The design of a mobile base 49 (FIG. 6) with deployable stabilizing stand 63 resolves the conflict between criteria for mobility and stability. In addition, the supporting point of the support stand 63 can vary along the base stem 61 with respect to the elevation of the manipulation. This makes stability adjustment independent of the structure 13 height. As such the designed base 49 is less space intrusive compared to a conventional tripod base. This is an important operation requirement in the space constrained surgical theater.

Accuracy of needle insertion is usually compromised by uncertainties like tissue deformation, needle deflection, motion of subject due to respiration or heartbeats, and registration misalignment. Respiration gait compensation can be integrated readily to the planning model at 33 (FIG. 2) and registration misalignment can be resolved by selection of higher resolution equipment. Of all uncertainties, modeling deformation in organs is the most challenging task. This is due to the complexity associated with the modeling of soft tissue, organ geometry and boundary constraints. Soft tissue is usually inhomogenous, nonlinear, anisotropic, and viscoelastic. The patient specific nature of soft tissue behavior also renders generalized soft tissue properties inaccurate for tissue modeling. Moreover it has been shown that organ geometry and boundary constraint are the dominant factors for deformation. This is a topic of great research interest. It is however, independent to Applicant's design centric objective as a predictive model can be easily integrated into the ablation planning algorithm once a reliable organ deformation model is available.

Apart from transcutaneous needle insertion, the robotic system 100 can also perform ablation treatment in laparoscopic procedures. There may be instances where a transcutaneous procedure causes potential complications due to the location of the tumor or the limited task space capacity of the manipulator 13. The surgeon may decide to do a laparoscopic procedure if the needle 47 placement options risk inevitable burning of neighboring organs or the abdominal wall. In such situations the ability to execute software controlled RCM facilitates multiple needle insertions to treat relatively large tumor areas. The needle 47 can be pivoted remotely at an isocentric point constrained by the incision port.

A computation scheme to derive the inverse kinematics is presented. This computation method is designed for the proposed task-specific needle insertion for laparoscopic mode of operation. The computation method assigns the global frame of reference at the constrained entry point. For a given set of target insertion points, a corresponding set of end-effector 45 positions can be obtained. The end effector position refers to the position of Frame 7 (conventional frame assignments according to mobility of the needle 47) with respect to the global frame at the port. Hence joint coordinates (q₄, qₛ, q₆, q₇, q₈) can be computed such that the needle 47 is constrained within the entry port. The computational scheme is presented as follows:

Step 1: Compute end-effector position

For a given position of target P_{T}(x_{T}, y_{T}, z_{T}), find end-effector 45 position P_{E} (x_{E}, y_{E}, z_{E}).

Since our application uses a rigid needle 47, the corresponding end-effector 45 positions can be obtained from the target points with the following transformation as shown in equation (1).

P_{E}=−IₖP_{T} (1)
where

$${I}_{k} = [ \begin{matrix}k & 0 & 0 \\ 0 & k & 0 \\ 0 & 0 & k\end{matrix} ]$$

is the scaling matrix and

$$k = \frac{{z}_{E}}{{z}_{T}}$$

z_{E} is the Z-axis coordinates of the end effector 45 and is a constant determined during the deployment of the submanipulator 43 discussed previously. The negative scaling matrix effectively maps the target position to the opposite octant of the 3D Cartesian space. This mapping relationship applies to the transformation of the distal end to the proximal end of any general rigid laparoscopic tools attached to the end effector 45.

Step 2: Obtain translational joint coordinate q₄ and q₅

By geometric inspection of the manipulator 13 design, the following relationship of the joint and task coordinates can be observed.

$$( \begin{matrix}{q}_{4} \\ {q}_{5}\end{matrix} ) = ( \begin{matrix}- {y}_{E} \\ {x}_{E}\end{matrix} ) \qquad ( 2 )$$

Step 3: Obtain joint coordinate q₆, q₇ and q₈

As discussed previously, the remote center of motion can be analyzed as a multibody system with four degrees of freedom. For analyzing needle application, the orientation along the axial direction of the needle 47 shaft is not relevant. Hence the motion of the end effector 45 with respect to the global frame is analyzed as three degrees of freedom motion including rotation along X and Y axes, and translation along the axial direction of the needle 47 shaft. Mathematically, this can be represented by a homogenous transformation matrix shown in equation (3).

$${T}_{E} = {R}_{x} ( α ) {R}_{y} ( β ) {P}_{z} ( r ) = [ \begin{matrix}c β & 0 & s β & rs β \\ s α s β & c α & s α c β & - rs α c β \\ - c α s β & s α & c α c β & rc α c β \\ 0 & 0 & 0 & 1\end{matrix} ] \qquad ( 3 )$$

where Rk(θ) is a rotation of θ about k-axis and P_{z}(r) is a translation of r along Z-axis.

By comparing the position coordinates in the transformation matrix, the following expressions can be obtained.

$$\begin{matrix}r = \sqrt{{x}_{E}^{2} + {y}_{E}^{2} + {z}_{E}^{2}} & ( 4 ) \\ β = arcsin ( \frac{x}{{q}_{8}} ) & ( 5 ) \\ α = arctan ( - \frac{y}{z} ) & ( 6 )\end{matrix}$$

This coordinate system is essentially a spherical coordinate system and can be related to our generalized joint coordinate system of 35 (FIG. 2) as follows.

$$( \begin{matrix}{q}_{6} \\ {q}_{7} \\ {q}_{8}\end{matrix} ) = ( \begin{matrix}α \\ - β - \frac{π}{2} \\ r\end{matrix} ) \qquad ( 7 )$$

A computation scheme to obtain the relative transformation matrix from the incision point to the target point can be computed as illustrated below.

Step 1: Obtain transformation matrix of target point with respect to fiducial marker frame, ^{M}T_{T}

^{M}T_{T}=[K]^{M′}T_{T′} (8)

M and M′ denote frame assigned to the fiducial marker in world coordinates and image coordinates respectively. T and T′ denote frame assigned to target point in world and image coordinates.

Step 2: Obtain transformation matrix of incision port with respect to fiducial marker frame, ^{P}T_{M}

The principle is to acquire a geometrical relationship of the incision port and the fiducial marker.

^{P}T_{M}=^{P}T_{G}^{G}T_{M} (9)

where P denotes frame assigned to incision port,
M denotes frame assigned to fiducial marker, and
G denotes a chosen global frame.

Step 3: Obtain transformation matrix of target point with respect to incision port, ^{P}T_{T}

^{P}T_{T}=^{P}T_{M}^{M}T_{T} (10)

The coordinates are obtained from the 3D tracking system reference to a global frame.

While this invention has been particularly shown and described with references to example embodiments thereof, it will be understood by those skilled in the art that various changes in form and details may be made therein without departing from the scope of the invention encompassed by the appended claims.

Other embodiments are further provided in “A Robotic System for Overlapping Radiofrequency Ablation in Large Tumor Treatment” by L. Yang, et al. in IEEE/ASME Transactions on Mechatronics, Vol. 15, No. 6, December 2010 (pgs. 887-897) a copy of which appears as Appendix I of the priority U.S. Provisional application herein incorporated in its entirety. Other details for embodiments are provided in Appendix II of the priority U.S. Provisional application herein incorporated in its entirety.

What is claimed is:

1. A method of computer-aided surgery having robot assisted needle insertion, comprising:
preoperatively imaging a target tumor area and planning target needle positions, resulting in a preoperative treatment plan;
using a 3D coordinate tracking system, translating the preoperative treatment plan into intraoperative execution, wherein the preoperative treatment plan has target needle positions in image coordinates, and the translating (i) transforms target needle positions to real world coordinates and (ii) digitally computes a joint trajectory plan by converting a design specific kinematic model for treating the target tumor area to robotic paths; and
robotically executing transcutaneous insertion of a needle following the robotic paths of the joint trajectory plan.

2. A method as claimed in claim 1 wherein the step of robotically executing is by a robotic manipulator and the 3D coordinate tracking system.

3. A method as claimed in claim 2 wherein the step of robotically executing includes:
confirming validity of placement of a first needle insertion; and
using the confirmed valid first needle placement as a reference for subsequent needle insertions.

4. A method as claimed in claim 2 wherein the robotic manipulator is of micro-macro manipulator design.

5. A method as claimed in claim 2 wherein the robotic manipulator employs mechanical linkage sensory units.

6. A method as claimed in claim 5 further comprising providing surgical navigation by including an optical registration system in tandem with the mechanical linkage sensory units.

7. A method as claimed in claim 1 wherein the target tumor area is a sufficiently large tumor area requiring multiple ablations, and the method performs overlapping ablation techniques.

8. A method as claimed in claim 1 wherein the robotic execution delivers transcutaneous ablation therapy.

9. A computer-aided surgery system having robot assisted needle insertion, comprising:
a 3D coordinate tracking system; and
a robotic needle insertion device providing intraoperative navigational guidance for needle placement, and
a processor in communication with the robotic needle insertion device, wherein a preoperative treatment plan is formed based on images of a target and initial planned target needle positions, the processor using the 3D coordinate tracking system and translating the preoperative treatment plan into intraoperative execution by the robotic needle insertion device,
wherein the preoperative treatment plan has target needle positions in image coordinates, and the processor translating includes (i) transforming target needle positions to real world coordinates and (ii) digitally computing a joint trajectory plan by converting a design specific kinematic model to robotic paths, in a manner enabling the robotic needle insertion device to robotically execute transcutaneous insertion of a needle following the robotic paths.

10. A computer-aided surgery system as claimed in claim 9 wherein the robotic needle insertion device comprises a robotic manipulator.

11. A computer-aided surgery system as claimed in claim 10 wherein:
the processor confirms validity of placement of a first needle insertion; and
the robotic manipulator uses the confirmed valid first needle placement as a reference for subsequent needle insertions.

12. A computer-aided surgery system as claimed in claim 10 wherein the robotic manipulator is of micro-macro manipulator design.

13. A computer-aided surgery system as claimed in claim 10 wherein the robotic manipulator employs mechanical linkage sensory units.

14. A computer-aided surgery system as claimed in claim 13 further comprising an optical registration system in tandem with the mechanical linkage sensory units for surgical navigation by the robotic needle insertion device.

15. A computer-aided surgery system as claimed in claim 9 wherein the target is a sufficiently large tumorous area requiring multiple ablations, and the robotic needle insertion device performs overlapping ablation techniques.

16. A computer-aided surgery system as claimed in claim 9 wherein the robotic needle insertion device delivers transcutaneous ablation therapy.
